(19) United States
(12) Patent Application Publication
(10) Pub. No.: US 2006/0016125 A1
(43) Pub. Date: Jan. 26, 2006

(54) Light treatment for reduction of tobacco specific nitrosamines

(75) Inventors: Marc Robert Krauss, Midlothian, VA (US); Qinglin Li, Richmond, VA (US); Dianne B. Jennings, Midlothian, VA (US)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: PHILIP MORRIS USA INC.

(21) Appl. No.: 11/184,851

(22) Filed: Jul. 20, 2005

Related U.S. Application Data
(60) Provisional application No. 60/590,419, filed on Jul. 23, 2004.

(51) Int. Cl. A01N 65/00; A01B 79/00; A61K 36/81

(52) U.S. Cl. 47/58.1 R; 424/751

(57) ABSTRACT

Methods of producing tobacco having reduced levels of tobacco-specific nitrosamines by treating growing or fresh-cut tobacco with light, preferably comprising UV-C wavelengths, are described.

### BACKGROUND OF THE INVENTION

1. Field of the Invention

The invention relates to methods of producing cured tobacco with reduced levels of tobacco specific nitrosamines, and tobacco produced using such methods.

2. Description of the Related Art

Tobacco-specific nitrosamines (TSNAs), such as N-nitrosonomicotine (NNN) and 4-(methylnitrosamino)-1-(3-pyridyl)-1-butanone (NNK), can be found in air-cured and flue-cured tobacco. See, “Effect of Air-Curing on the Chemical Composition of Tobacco.” (Wiernik et al., Recent Adv. Tob. Sci., 21:39-80) According to Wiernik et al., TSNAs are not present in significant quantities in growing tobacco plants or fresh cut tobacco (green tobacco), but are formed during the curing process. Commonly owned U.S. patent application Ser. No. 10/235,636 by Krauss et al. (published Mar. 27, 2003 with publication no. 20030056801) describes a method for the reduction of tobacco specific nitrosamines by increasing antioxidants in tobacco. The methods disclosed include root pruning or severing the xylem tissue of the tobacco plant prior to harvesting. U.S. patent application Ser. No. 10/235,636 is incorporated herein by reference in its entirety for all purposes.

### SUMMARY

A method comprising treating tobacco plants with light, preferably comprising exposing the tobacco to UV-C light, prior to and/or after harvest can reduce the levels of tobacco-specific nitrosamines in cured tobacco.

In a preferred embodiment of the method, tobacco plants or fresh cut tobacco are exposed to doses of light, preferably comprising UV-C light. In more preferred embodiments, the light is of sufficient intensity and duration to cause an increase in the levels of antioxidants in the tobacco leaves. In a further preferred embodiment, the exposure comprises exposing the tobacco for at least about 10 minutes, for example at least about 15 or 30 minutes, preferably between about 15 minutes to about 2 hours. Depending on the intensity of the light, longer or shorter exposure times may be used, for example at least about 0.1, 0.15, 0.2 or about 2.5 hours or longer. Preferably, the source of light used in the treatment provides light having a peak intensity in the range of about 240 to 260 nm, for example, light having a peak or substantial component of intensity at about 254 nm or shorter is most preferred. However, in addition to UV-C wavelengths, a light source used in the method may also provide UV-B, UV-A and/or visual light.

Preferably, the method comprises treating the tobacco with light within the period about 7 days prior to about 72 hours after harvest, more preferably in the period from about 3 days prior to harvest to about 48 hours after harvest. Most preferably, treatment may be performed in the period after harvest and before the tobacco curing process begins in a curing barn, such as from immediately after harvest to about 48 hours later, for example up to about 24 hours after harvest.

In further exemplary embodiments, light treatment occurs one or more times prior to harvest, for example within about 3 weeks before harvest, within about 2 weeks before harvest, within about 1 week before harvest, or more particularly about 21, 17, 14, 12, 10, 9, 8, 7, 6, 5, 4, 3, 2 and/or 1 day or less before harvest and/or within about 1, 2 or 3 days after harvest. In addition to the above treatments, it may be desirable to expose tobacco to UV-C light one or more times during the curing process.

In preferred embodiments, treatment may comprise a single treatment. Alternatively, a series of 2 or more light treatments may occur at selected intervals. The intensity and/or duration of a series of treatments may be uniform or may vary, for example a series of lower intensity and/or shorter duration treatments may be followed by one or more higher intensity and/or longer duration treatments; alternatively, one or more higher intensity or longer duration treatments can be followed by one or more shorter duration and/or lower intensity treatments. As an example of such embodiments, the tobacco plants can be acclimated to a series of lower intensity and/or shorter duration treatments and one, or more, higher intensity and/or longer duration treatments may be applied in a period closer to harvest or within about 2 days after harvest.

Un-cured tobacco treated as described above preferably has increased levels of antioxidant activity and/or capacity relative to untreated tobacco. Cured tobacco produced by methods comprising the treatments described above preferably has substantially reduced levels of one or more TSNAs relative to untreated tobacco.

### BRIEF DESCRIPTION OF THE DRAWINGS

FIG. 1 shows that the water soluble antioxidant capacity, as measured by the ability of leaf extracts to quench superoxide, increased after UV-C treatment. Variation in the overall capacities seen between the two treatments may be attributed to treatments being applied on different days. Data shown are the means of four plants at each time point±standard error (S.E.).

FIG. 2 shows that UV-C treatment resulted in increased lipophilic antioxidant capacities (AOC) above basal levels. However, with the high UV-C dosage antioxidant capacity returned to basal levels by 48 hrs after treatment. This decrease may be the result of damage to the leaf tissues at the high dosage. Data shown are the means of four plants at each time point±S.E.

FIGS. 3A and 3B show chlorogenic acid (CGA), rutin and scopoletin concentrations in untreated and treated tissues that were analyzed by HPLC. Scopoletin was low or undetectable in all samples (data not shown). Rutin and CGA changed with UV-C exposure. 48/72 hrs after UV-C treatment CGA concentrations were elevated above basal levels in plants exposed to low and high dosages. Rutin increased during the 72 hr period after low UV-C exposure. At the higher UV-C dosage the increase in rutin was transitory and concentrations were lower than control by 48 hrs. Data shown are the means of four plants at each time point±S.E.

FIGS. 4A and 4B show that analysis of two lipophilic antioxidants by HPLC indicated differing responses of β-carotene and α-tocopherol to UV-C exposure. Concentrations of β-carotene increased in plants subjected to low or high doses of UV-C. However, α-tocopherol concentrations appeared to decrease in the same plants. While the increased β-carotene may account for some of the increased lipophilic antioxidant capacity (AOC) noted, there are likely other antioxidants and/or synergistic/antagonistic interactions that contribute to overall AOC. Data shown are the means of four plants at each time point±S.E.

FIG. 5 illustrates that when tobacco plants were treated with UV-C light there was a decrease in photosynthetic rates 24 hr after treatment. However, photosynthetic rates returned to control levels by 48 hrs after UV-C exposure. Data represent the average of four plants per treatment, three leaves per plant±S.E.

FIGS. 6A and 6B shows that altered photosynthetic rates corresponded to changes in the light absorbance spectra of the leaves of high and low dosage UV-C exposed plants. Graphs are representative of plants before (upper line) and 24 hrs after (lower line) the UV-C exposure.

FIG. 7 shows that phenylalanine ammonia lyase activity of UV-C treated plants increased in a dose dependent manner. These increases were transient, rates returning to control levels by 48/72 hrs after UV-C treatment.

FIG. 8 illustrates downstream products of the phenylalanine ammonia lyase pathway.

FIG. 9 illustrates the chamber used in Example 2 to expose tobacco plants to UVC light after harvest.

FIG. 10 compares reduction in total TSNAs in cured tobacco that was treated with approximately 60 mW/cm² of light with a peak intensity at 254 nm (UV-C) for 15 minutes immediately after harvest versus 3 days after harvest.

FIG. 11 compares reduction in total TSNAs in cured tobacco treated within 24 hours of harvest with approximately 60 mW/cm² of light with a peak intensity at 254 nm (UV-C) for 10 minutes versus 15 minutes.

FIGS. 12 and 13 show example arrangements for apparatuses that can be used to treat harvested tobacco.

### DETAILED DESCRIPTION OF THE PREFERRED EMBODIMENTS

Without being bound by theory as to the mechanism of the effect of the treatments described herein, it is known that a wide range of plant defense responses are induced by oxidative stress, including increases in antioxidant activity. Major antioxidants present in plant tissue are phenols, ascorbic acid, tocopherol and carotenoids. Antioxidants interfere with oxidation through chain-breaking reaction processes or through scavenging of free radicals. For many land plants, the production of antioxidants in leaves can be induced or up-regulated by exposure to stressful conditions. The reactive oxygen species produced under stress conditions act as a stress signaling agent to activate defense mechanisms. Responses include the cross linking of cell wall proteins, the activation of protein kinases, and the increased expression of various plant protectant and defense genes. Some of these genes encode peroxidase, glutathione S-transferase, proteinase inhibitors, and various biosynthetic enzymes, such as phenylalanine ammonia lyase (PAL). PAL is the first enzyme in the phenylpropanoid pathway, which is involved in the synthesis of polyphenols and flavonoids. Model studies have shown that antioxidants such as ascorbic acid, polyphenols, flavonoids and cysteine inhibit the formation of nitrosamines. (Rundlöf et al., J Agric Food Chem., 48:4381-8, 2000).

Ultraviolet (UV) radiation is electromagnetic radiation of a wavelength shorter than that of visible light, but longer than that of soft X-rays. The range of UV wavelengths is often subdivided into UV-A (380-315 nm), UV-B (315-280 nm), and UV-C (280-210 nm). Ordinary glass is transparent to UV-A, but is opaque to shorter wavelengths. Quartz glass, depending on quality, can be transparent to UV wavelengths. Because of absorption in the atmosphere's ozone layer, 99% of the ultraviolet light that reaches the Earth's surface is UV-A.

Exposure to high intensity light, for example UV-C light of sufficient intensity, can produce an oxidative stress reaction in plants. Increases in antioxidant activity in response to UV-C light have been reported in in vitro laboratory experiments in soybean cotyledons and tobacco callus in a tissue culture context. Kozak et al. reported antioxidant response of soybean cotyledons (Glycine max) to ultraviolet radiation. (Canadian Journal of Plant Science, 79:171-89, 1998). After 24 hours of UV-B and UV-C irradiation, homogenates of superficial layers of soybean cotyledons were reported to contain significantly more tocopherol and ascorbic acid. Zacchini and De Agazio have also recently reported analysis of tobacco callus cultures exposed to UV-C high dose pulse-treatment. After 6, 24 and 48 h from the end of the treatment, oxidative damage (malondialdehyde and hydrogen peroxide), non-enzymatic (radical scavenging antioxidants and polyamines) and enzymatic antioxidants (ascorbate peroxidase, glutathione reductase, catalase and guaiacol peroxidase) were evaluated. A strong increase of H₂O₂ content and a slight cellular damage was observed 24 and 48 h after UV-C treatment. Initially, activation of non-enzymatic antioxidants, followed by activation of enzymatic antioxidants, was detected. (Plant Physiol Biochem. 42:445-50, May 2004).

Increased levels of antioxidant in fresh cut tobacco interfere with the production of TSNAs, thereby resulting in lowered TSNA concentrations in cured tobacco. By treating growing tobacco plants with a sufficient dose of UV-C light in the about 7 weeks before harvesting, for example within about 3 weeks (i.e. 2, 3, 4, 5, 6, 7, 8, 9, 10, 11, 12, 13, 14, 15, 16, 17, 18, 19, 20, 21, 22, 23, 24, or 25 days) prior to harvesting, such as about 1-3 weeks after topping (i.e. cutting of the apex of the plant), an increase in antioxidant activity in the fresh cut tobacco can be obtained. Preferably, treatment comprises an exposure within about 1 week prior to harvest and/or within about 48-72 hours after harvest. This can result in significant reductions of TSNA production during curing (i.e., air-curing, flue-curing, fire-curing, sun-curing, and any other means of curing known or contemplated by one of skill in the art) of the tobacco. It should be noted that while changes in antioxidant have been observed in some cases, reductions of TSNA production during curing have also been observed to result from treatments as described herein even where changes in antioxidant activity were transient or not observed. Thus, observable changes in antioxidant activity, although a preferable effect in some circumstances, should not be considered a prerequisite for effective treatment resulting in reductions of TSNA production during curing.

Treating with a dose of light means exposure to a selected intensity of light comprising substantial intensity at selected wavelengths for a selected period. Approximately equivalent doses may comprise high intensity light for a short period or lower intensity light for longer periods. Preferably, the dose is effective to increase the concentration and/or capacity of antioxidant in the tobacco leaves at 24, 48 and/or 72 hours after exposure. An increase in antioxidant means at least a statistically significant increase (i.e. at least 95% confidence) compared to untreated material. Concentration and capacity of antioxidants in tobacco leaves can be measured by any recognized method, such as described in the examples below.

After harvest, a tobacco plant's metabolic processes continue for a period of time that depends on conditions such as temperature and condition of the plant at harvest. An increase in antioxidant activity or capacity in fresh cut tobacco may also be obtained by exposing the fresh-cut tobacco to sufficient intensity and duration of UV-C light shortly after harvesting but prior to curing, for example in the period from 0 to about 72 hours after harvest, or more preferably 0 to about 48 hours after harvest, for example within about one day of harvest. In addition, by treating the tobacco in the period shortly after harvest and before curing, any disruption to traditional growing and harvesting practices may be minimized. Moreover, treatment in the period after harvest but prior to curing may also take advantage of the anti-microbial properties of UV-C light to reduce populations of nitrosamine producing microbes on the tobacco. Accordingly, a method comprising treating tobacco plants with light, preferably comprising exposing the tobacco to UV-C light, prior to and/or after harvest can result in cured tobacco having reduced levels of TSNAs. If desired, harvested tobacco can also be exposed to UV-C light at one or more points in the curing process.

Exposing the plants to light comprising UV-C wavelengths in the field can be accomplished by use of portable lights, pole-mounted flood-lights, boom-mounted lights or mobile lights, for example lights mounted on a tractor or lights mounted on irrigation equipment. Fresh-cut tobacco may be exposed to UV-C light using standard lighting fixtures mounted on apparatus adapted for the purpose, for example hanging tobacco may be placed in a chamber which positions a 15 watt UV-C light about 24 inches from the leaves, or fresh cut tobacco may be placed on a conveyor that moves the tobacco past one or more bulbs arranged to deliver a desired dosage.

Preferred dosages of UV-C light, i.e. light having a peak intensity at about 254 nm, have an intensity of light at the tobacco leaf of preferably between about 1-400 mW/cm², more preferably between about 1-100 mW/cm², for example from about 1-60 mW/cm² or more preferably about 14-60 mW/cm² for periods of about 1 minute for the more intense light to as long as about 120 minutes for the least intense light, or about 15 to about 60 minutes. Most preferably the intensity and period are about 15 to about 60 minutes for light intensities of about 1 to about 60 mW/cm² or more preferably about 14-60 mW/cm². Effective periods of exposure depend inversely on the intensity of light and vice versa. Further, over exposure can damage the plant, which may not be desirable. For example exposure to UV-C light at an intensity of about 400 mW/cm² or more for about 30 minutes or less can damage the plant. One skilled in the art may choose higher or lower intensities than listed above combined with shorter or longer periods of exposure than listed above to achieve a desired effect with a given light source.

To provide an illustrative example, an intensity of 1 to 60 mW/cm² can be obtained by placing a 15 W UV-C bulb, such as the bulb sold by Spectroline as Model XX-15F, about 2 feet from a tobacco plant. At this intensity, the tobacco can preferably be exposed for about 15 minutes to obtain an effective dose. In various embodiments of the method, such a bulb could be placed about 1 to 3 feet from the tobacco to be treated for about 5-60 minutes, generally less than one hour, to achieve an effective dosage. Placing such a 15 W UV-C bulb within about 6 inches results in exposure of greater than about 400 mW/cm², which can cause damage in about 30 minutes or less. Of course, one skilled in the art can modify these distances and times to achieve an effective dose using various light sources of different intensities and spectral properties.

Light comprising UV-C wavelengths for use in these methods, is preferably light having a peak intensity in the range about 240-260 nm, for example light having a peak intensity at about 254 nm. Depending on the light source used to produce the UV-C light, the light may have additional peaks in other areas of the light spectrum. For example, the light may also comprise UV-B, UV-A and/or visual light in addition to the UV-C light. Antimicrobial properties of UV-C light may be exploited by exposing curing tobacco to light comprising UV-C light during curing in barns and/or processing facilities.

In a preferred embodiment of the method, tobacco plants are exposed to doses of light, preferably comprising UV-C light, of sufficient intensity and duration to cause an increase in the levels of antioxidants in the tobacco leaves in addition to other changes. For example, the level of phenylalanine ammonia lyase activity in treated plants can be increased by about 4 to about 16 fold at about 24 hours after treatment.

In preferred embodiments, treatment may comprise a single treatment or a series of 2 or more treatments at selected intervals. The intensity and/or duration of such a series of treatments may be uniform or may vary, for example a series of lower intensity and/or shorter duration treatments may be followed by one or more higher intensity and/or longer duration treatments; alternatively, one or more higher intensity or longer duration treatments can be followed by one or more shorter duration and/or lower intensity treatments. As an example of such embodiments, the tobacco plants can be acclimated to a series of lower intensity and/or shorter duration treatments while growing and one, or more, higher intensity and/or longer duration treatments may be applied in a period closer to harvest, for example about 24 to 72 hours prior to harvest. Thus, in various embodiments, the method may comprise acclimating growing tobacco to UV-C light treatments over a period prior to harvest and optionally to utilizing a final treatment prior to or after harvest, such as about 24 to 72 hours prior to harvest or within about 24 or 48 hours after harvest.

Light treated tobacco can be cured by any suitable method such as methods that are conventional in the art. Conventional air-curing tobacco barns typically utilize natural convection, with air flow generally proceeding from the bottom of the barn toward the top of the barn. In curing tobacco by the procedure generally referred to as the “bulk curing” method, tobacco leaves are typically loaded in a relatively compact mass on racks or in containers and placed inside of an enclosed curing barn where a furnace or a plurality of heaters circulate a forced flow of heated air through the mass of tobacco leaves to effect curing and drying. Conventional tobacco curing barns attempt to obtain the desired atmospheric conditions such as temperature and humidity within the tobacco barn by various adjustments of louvers or openings in the sides of the barn and the operation of heaters spaced along the floor of the barn with respect to the prevailing temperature and moisture content of the outside atmosphere, the wind velocity and its direction with respect to the tobacco barn.

It may alternatively be desirable to utilize a method for curing tobacco as described in commonly owned U.S. Pat. No. 6,786,220, which is incorporated by reference herein in its entirety. Briefly, such a preferred method for air curing tobacco includes the tobacco being hung in an enclosure having at least one vertically arranged air duct positioned in a central portion of the enclosure, at least one in-line fan positioned in a vertical portion of the at least one vertically arranged air duct, at least one ventilating fan located in an upper portion of the enclosure and at least one openable and closeable opening in at least one side wall of the enclosure, with the method including the steps of opening the at least one opening, and operating the at least one ventilating fan to force air down through the tobacco from the upper portion of the enclosure. The method of curing tobacco can include the steps of closing the at least one opening and introducing an aqueous solution or steam into a lower portion of the at least one vertically arranged air duct and operating the at least one in-line fan to diffuse the moisture and drive it upwards through the vertically arranged air duct.

Growing or fresh-cut (green) tobacco treated as described above preferably has increased levels of antioxidant activity relative to untreated tobacco, at least in the period 48 to 72 hours following treatment. Cured tobacco produced by methods comprising the treatments described above preferably has substantially reduced levels of one or more TSNAs relative to similarly grown and processed tobacco wherein the treatments were not applied.

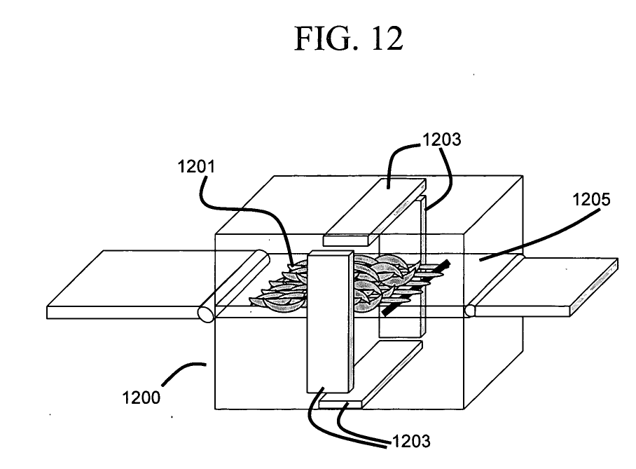

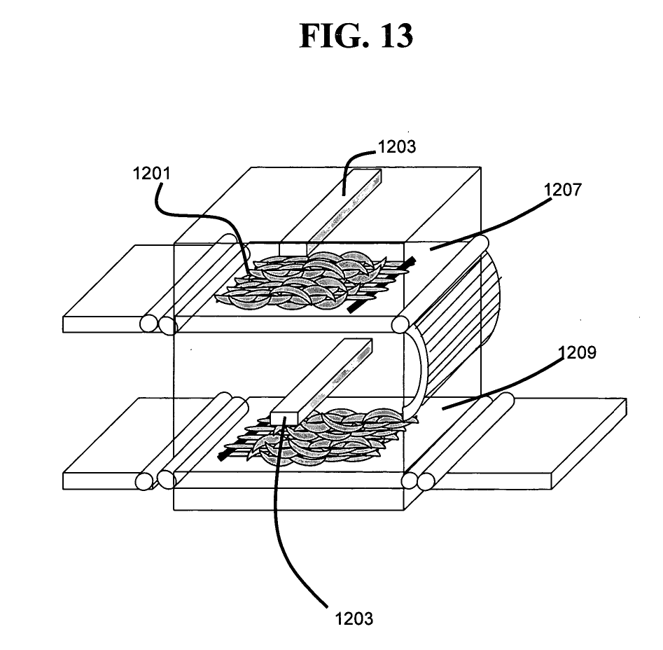

As illustrated in FIGS. 12-13, an apparatus for treating tobacco following harvest can comprise a chamber 1200 through which tobacco 1201 can be passed on a conveyor 1205 where it will be exposed to light from one or more light sources 1203. Preferably the light sources are UV-C lights, i.e. sources that provide light comprising UV-C wavelengths, more preferably the light sources provide light having substantial intensity or a peak intensity at about 254 nm. The tobacco may comprise one or more plants attached to a stick, or the tobacco may be placed directly on the conveyor. The lights are preferably arranged to provide substantially uniform coverage of the tobacco material and the conveyor speed can be adjusted to provide a desired dosage of the UV-C light.

A single pass can be used as illustrated in FIG. 12. A conveyor 1205 is preferably made of material such that UV-C light may pass through, permitting light sources 1203 above and below the tobacco to simultaneously illuminate both sides. Accordingly, conveyor 1205 can be a material which transmits light, or may comprise a mesh of plastic, cloth, metal or other suitable material. A system of multiple chambers in series, or an elongated chamber comprising multiple light sources along its length, can be used to permit faster rates of conveyance while maintaining a desired dose of the light. Alternatively, a system can be constructed of two or more chambers 1200 in series where the tobacco is treated on one side in each chamber and is manually or mechanically turned between chambers. In such cases, the conveyor can be any suitable material.

FIG. 13 illustrates an arrangement whereby tobacco can be passed on conveyors 1207 and 1209 through a chamber in opposing directions with the conveyors so that the tobacco is flipped as it passes from one to another. It will be appreciated that additional variations can be utilized. For example, tobacco that has been attached to sticks for hanging can be conveyed through a treatment chamber by an arrangement in which the sticks upon which tobacco plants are hung are carried vertically on a track or pair of tracks comprising movable elements arranged to engage each stick near the ends and to convey the hung tobacco through the chamber. In preferred embodiments, the apparatus or conveyors attached to the apparatus can be positioned so as to transport harvested tobacco into a tobacco curing barn or in the direction of an entrance thereof so that harvested tobacco can be treated with light according to the methods described herein as it is being transported into the curing barn.

While the invention has been described in detail with reference to preferred embodiments thereof, it will be apparent to one skilled in the art that various changes can be made, and equivalents employed, without departing from the scope of the invention. Further, while the following examples provide further illustration of the methods of the invention, these examples should not be considered limiting in any way.

### EXAMPLES

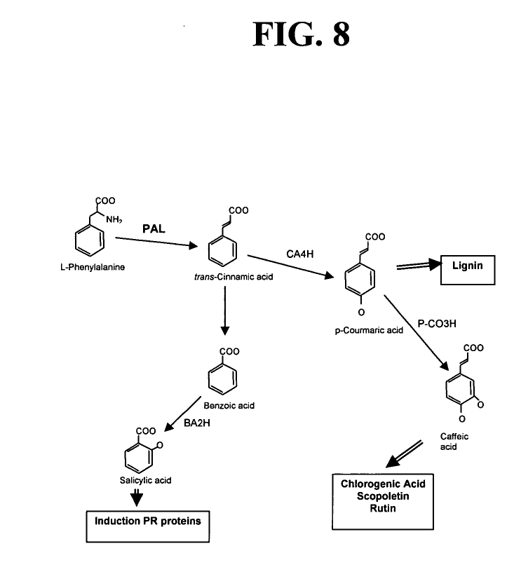

UV-C light (254 nm) is often utilized as a germicidal treatment. In nature UV-C is blocked by ozone and thus plants are not naturally exposed to it. However, when artificially exposed to UV-C a variety of defense responses are triggered in plants. (Mari, M. and Guizzardi, M. Phytoparasitica 26:59-66, 1998). Among these is an increase in phenylalanine ammonia lyase (PAL) a key enzyme in phenylpropanoid metabolism. (Lers, A., et al., Plant Mol. Biol., 36:847-856, 1998). As illustrated in FIG. 8, there are numerous downstream products that could be potentially altered by changes in PAL activity, including increases in salicylic acid, lignins, and antioxidants (chlorogenic acid, rutin, etc.).

### Example 1

A study was undertaken to observe dosage dependant effects of UV-C in inducing a stress response without damaging the plants and to measure changes in the antioxidant capacity of treated leaves. Photosynthetic rates, spectral reflectance measurements and PAL activity rates were observed to show the physiological status of the plants before and after UV-C exposure.

UV-C treatment: Mature greenhouse grown Burley tobacco plants were exposed to UV-C light (Spectroline, model XX-15F) for 30 minutes with one 15 W bulb (“LOW”, n=4) or for 1 hr with two 15 W bulbs (“HIGH”, n=4). Plants were rotated 180 degrees every 15 minutes for 30 min treatment, and 90 degrees every 15 min for the 1 hr treatment, so that leaves received relatively the same “dose”. Leaf samples were taken just prior to exposure as well as 24 and 72 hrs for LOW dose and 48 hrs after exposure for HIGH dose.

Physiological Measurements: Photosynthetic rates were measured using a LI-COR 6400 infra-red gas analyzer (LI-COR, Lincoln, Nebr.) prior to UV-C exposure, as well as 24 and 48/72 hrs after. Three leaves per plant were analyzed and data are expressed as % of control plant photosynthetic rates taken at the same time. Spectral reflectances were assessed at the same time periods using a FieldSpec Handheld Spectroradiometer (ASDI, Inc, Boulder, Colo.). PAL assays were performed as described by Singh et al. (J. Exp. Bot., 50:1619-1925, 1999). Protein concentrations were determined using a protein assay kit (Bio-Rad) with BSA as standard (Bradford, Anal Biochem., 72:248-25, 1976).

Antioxidant Analysis: Liquid nitrogen frozen leaf tissues were ground to a fine powder and extracted with either a solution of 50% acetone/50% water (water soluble extract) or 99% hexane/1% 2-propanol (lipid soluble extract). Antioxidant capacities were measured by a photochemiluminescence method using an automated instrument, Photochem (Analytik Jena AG, Germany). This method uses two separate protocols to measure water soluble and lipid soluble antioxidant capacities, and assays were performed according to the manufacturer's specifications.

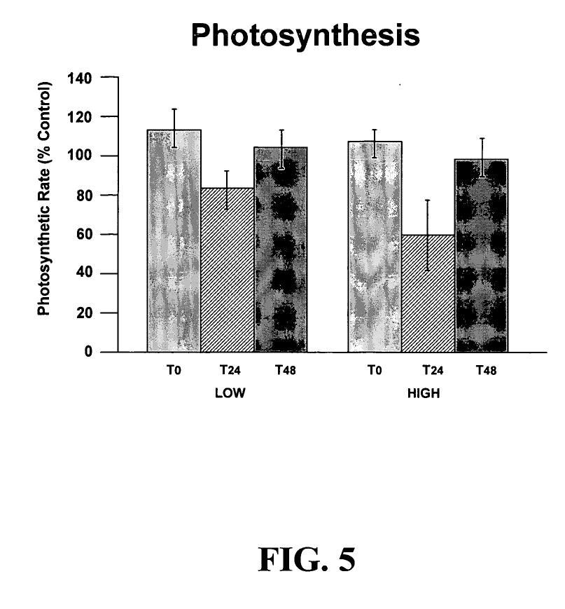

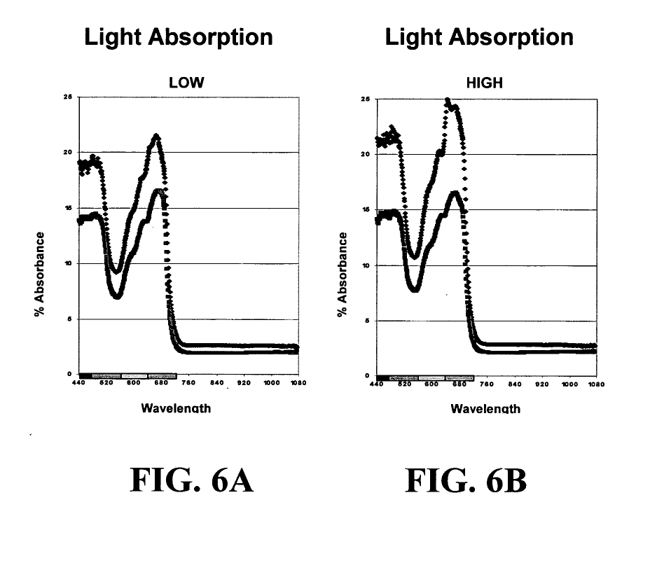

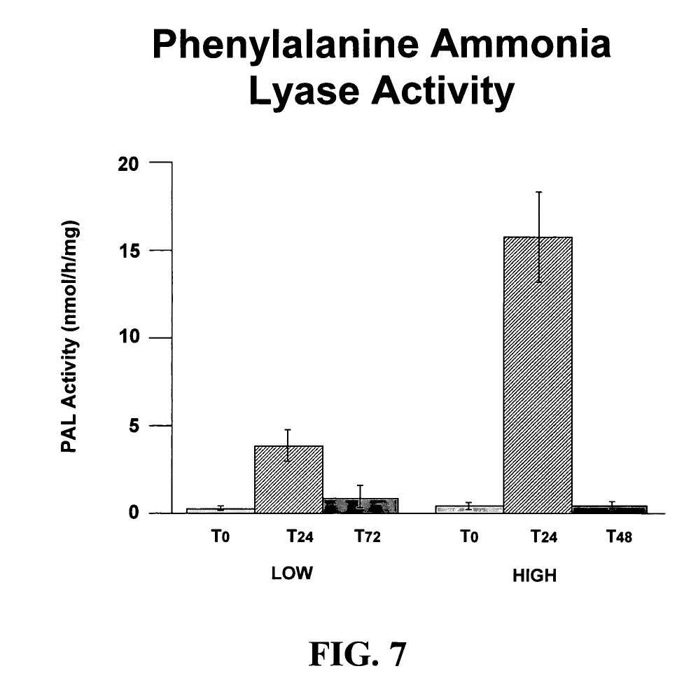

When tobacco plants were treated with UV-C light there was a decrease in photosynthetic rates 24 hr after treatment as shown in FIG. 5. However, photosynthetic rates returned to control levels by 48 hrs after UV-C exposure. Altered photosynthetic rates corresponded to changes in the light absorbance spectra of the leaves of UV-C exposed plants as shown in FIGS. 6A and 6B. Further, phenylalanine ammonia lyase activity of UV-C treated plants increased in a dose dependent manner as shown in FIG. 7. These increases were transient, rates returning to control levels by 48/72 hrs after UV-C treatment.

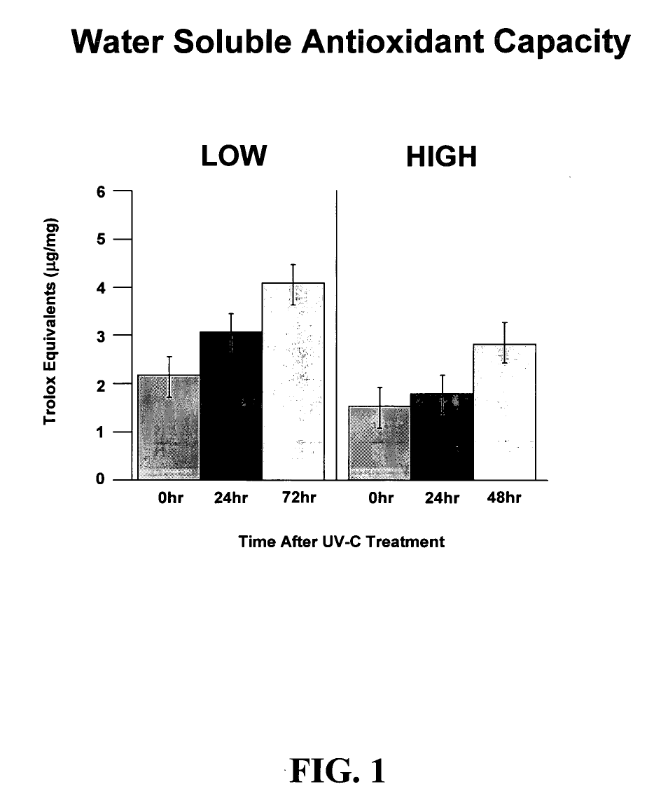

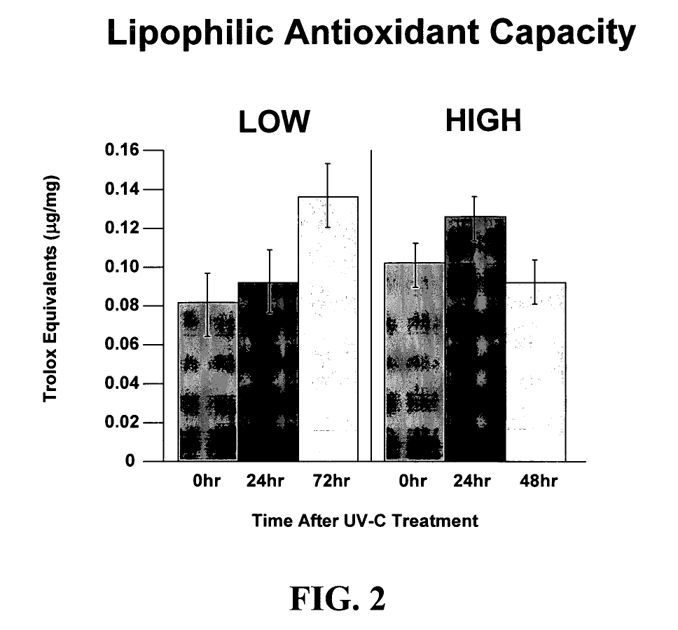

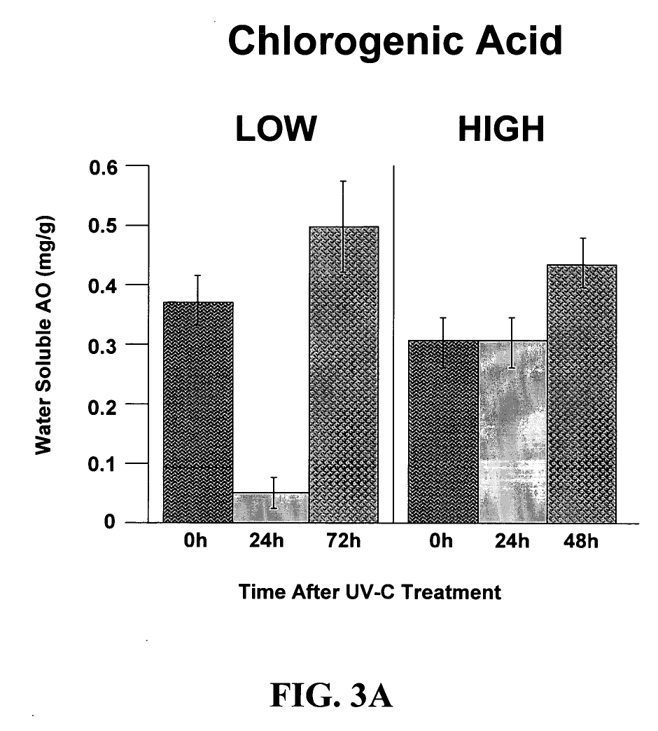

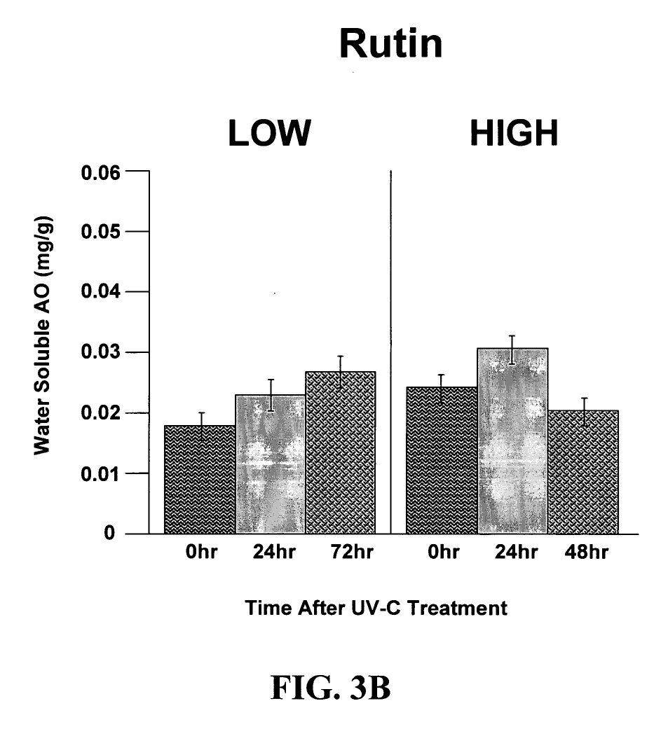

The water soluble antioxidant capacity, as measured by the ability of leaf extracts to quench superoxide, increased after UV-C treatment as shown in FIG. 1. Variation in the overall capacities seen between the two treatments may be attributed to treatments being applied on different days. UV-C treatment also resulted in increased lipophilic antioxidant capacities (AOC) above basal levels. However, with the high UV-C dosage antioxidant capacity returned to basal levels by 48 hrs after treatment as shown in FIG. 2. This decrease may be the result of damage to the leaf tissues at the high dosage. Chlorogenic acid (CGA), rutin and scopoletin concentrations in untreated and treated tissues were analyzed by HPLC as shown in FIGS. 3A and 3B. Scopoletin was low or undetectable in all samples (data not shown). Rutin and CGA changed with UV-C exposure. 48/72 hrs after UV-C treatment CGA concentrations were elevated above basal levels in plants exposed to low and high dosages. Rutin increased during the 72 hr period after low UV-C exposure. At the higher UV-C dosage the increase in rutin was transitory and concentrations were lower than control by 48 hrs.

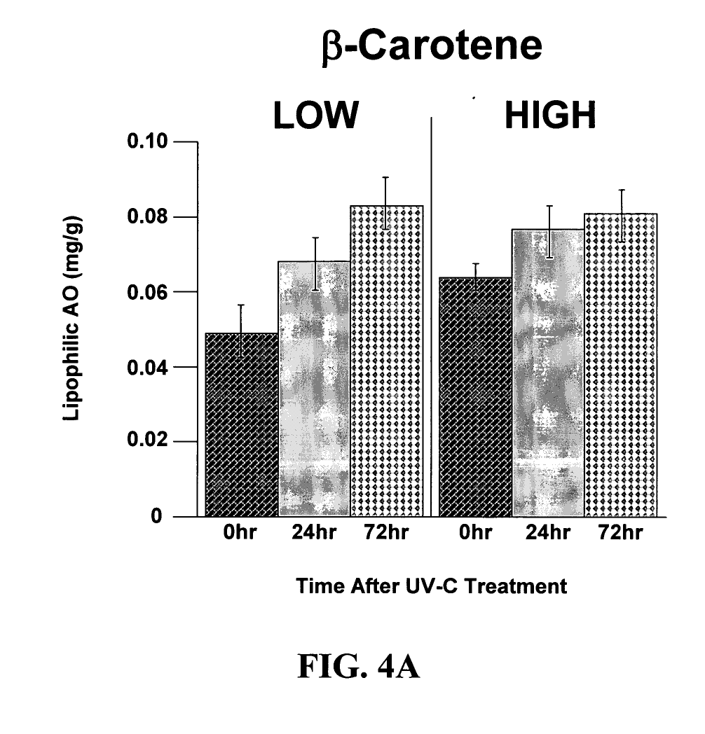

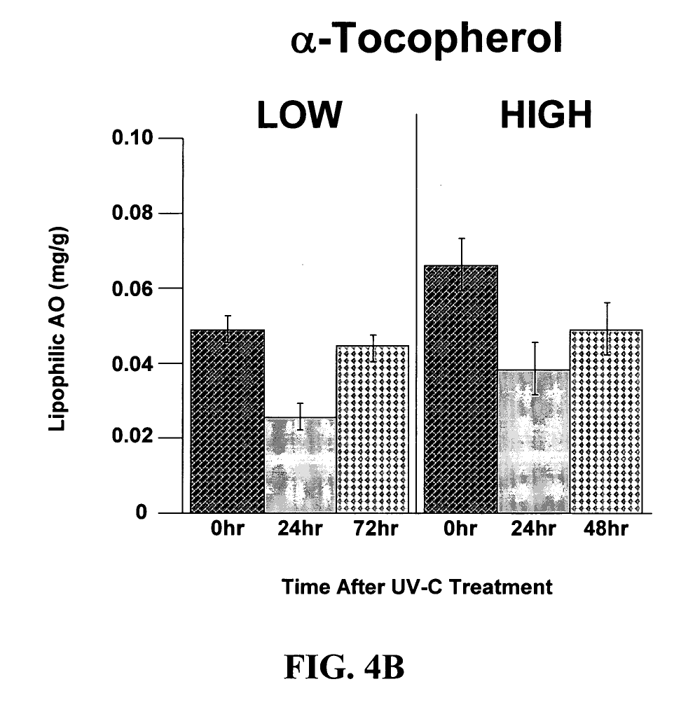

Analysis of two lipophilic antioxidants by HPLC indicated differing responses of β-carotene and α-tocopherol to UV-C exposure as shown in FIGS. 4A and 4B. Concentrations of β-carotene increased in plants subjected to low or high doses of UV-C. However, α-tocopherol concentrations appeared to decrease in the same plants. While the increased β-carotene may account for some of the increased lipophilic antioxidant capacity noted, there are likely other antioxidants and/or synergistic/antagonistic interactions that contribute to overall AOC.

At least the following can be discerned from these results. At both a “low” or “high” UV-C photosynthetic rates and light absorption spectra were dramatically altered. Absorbance spectra shifts indicate modifications in the abilities of the treated leaves to absorb photosynthetically. Both measures returned to pre-treatment conditions by 48 hrs after exposure. PAL activity increased, in a dose dependent manner, within 24 hrs of UV-C exposure but returned to control levels by 48-72 hrs after treatment.

At both UV-C “dosages” the water soluble antioxidant capacity increased up to 48-72 hrs. Chlorogenic acid concentrations increased over the measured time period at both exposure rates. Rutin concentrations increased with low UV-C treatment but the increase was not maintained over time at the high dosage. Low UV-C exposure resulted in increased lipophilic AOC. A transient increase was observed at the higher exposure. β-Carotene concentrations rose but α-tocopherol showed an overall decrease.

### Example 2

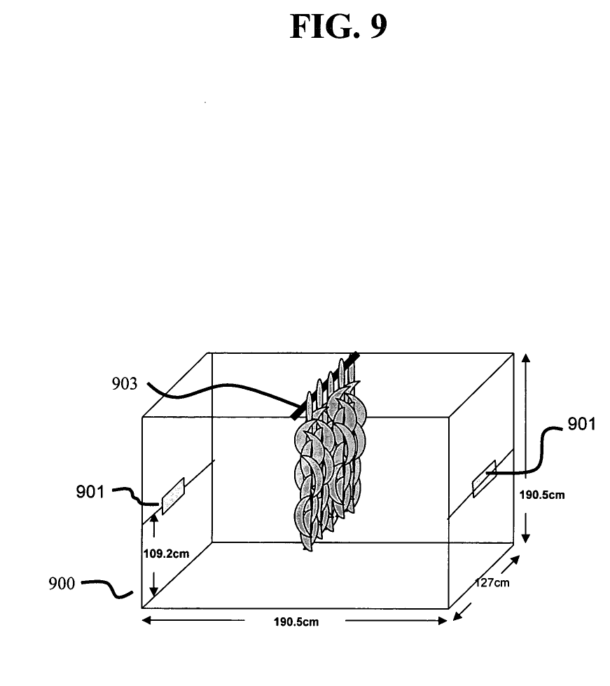

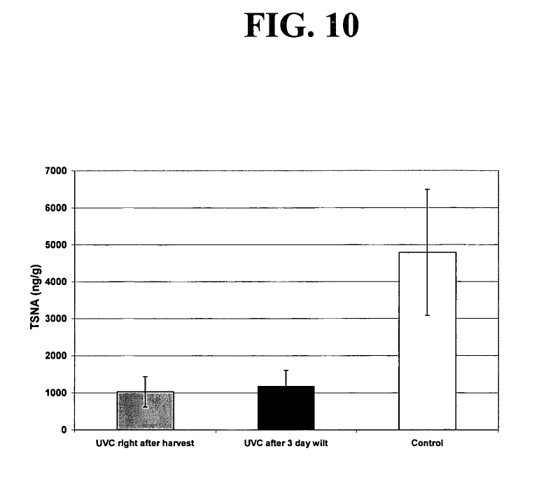

Harvested burley tobacco plants were treated with UV-C light either directly after harvest or three days after harvest (just prior to be put in the curing barn) using a chamber 900 as illustrated in FIG. 9. For both time points, sticks of harvested tobacco 903 (5 plants/stick) were treated with 15 minutes of UV-C light from two light sources 901 at opposite ends of the chamber 900. Both sides of each plant were exposed simultaneously to approximately 60 mW/cm² of irradiance having a peak intensity at 254 nm (UV-C). UV-C treated and control plants were cured in the same barn and TSNA concentrations were determined at the end of curing. Ten sticks of harvested tobacco plants were tested for each data point. A summary of the results are shown in FIG. 10.

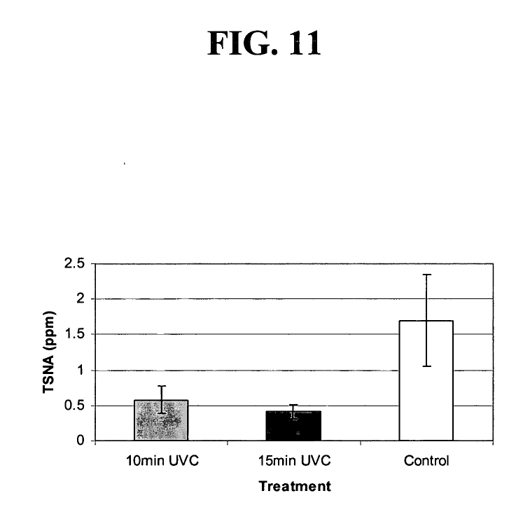

In another independent experiment, sticks of harvested tobacco were exposed to UV-C light within 24 hrs of harvest. Five sticks were irradiated (˜60 mW/cm²) for 10 minutes, and another five sticks were irradiated for 15 minutes. All sticks, including controls, were cured in the same barn and analyzed for TSNA, nitrates and nitrites following curing. A summary of the TSNA concentration results is shown in FIG. 11.

Treatment of harvested tobacco plants with UV-C light reduced TSNA formation during the curing process. The time of treatment, right after harvest or following a three day wilt, did not significantly impact the reduction of TSNAs attributable to UV-C treatment. Further, 10 minutes of UV-C light treatment at about 60 mW/cm² was substantially as effective at producing a reduction in TSNA concentration after curing as a 15 minute treatment. At the least, these data demonstrate reduction of TSNA formation in cured tobacco by treatment with UV-C light after harvest and prior to curing in two independent experiments conducted at different locations.

What is claimed is:

1. A method of producing cured tobacco having reduced levels of TSNAs, the method comprising:
treating tobacco comprising tobacco leaves with at least one dose of light having a spectrum comprising at least UV-C light prior to curing said tobacco.

2. The method of claim 1, wherein treating the tobacco comprises treating fresh-cut tobacco during the period within about 72 hours after harvest.

3. The method of claim 1, wherein treating the tobacco comprises treating fresh-cut tobacco during the period within about 48 hours after harvest.

4. The method of claim 1, wherein treating the tobacco comprises treating fresh-cut tobacco within about 1 day after harvest.

5. The method of claim 1, wherein treating the tobacco comprises treating growing tobacco plants one or more times in the period of about 7 weeks prior to harvest.

6. The method of claim 1, wherein treating the tobacco comprises treating growing tobacco plants within about 1 week prior to harvest.

7. The method of claim 1, wherein treating the tobacco comprises treating growing tobacco plants within about 3 days prior to harvest.

8. The method of claim 1, wherein treating the tobacco comprises a series of treatments with UV-C light.

9. The method of claim 1, wherein treating tobacco comprises a series of treatments with UV-C light prior to harvest.

10. The method of claim 1, wherein the light comprises a peak intensity at about 254 nm.

11. The method of claim 10, wherein the dose of light comprises light having an intensity of between about 1 to 60 mW/cm² at the surface of the tobacco leaf for a period between about 10 minutes to about an hour.

12. The method of claim 1, further comprising treating the tobacco with UV-C light one or more times during the curing period.

13. A method of producing cured tobacco having reduced levels of TSNAs, the method comprising,
harvesting tobacco comprising tobacco leaves,
treating the freshly harvested tobacco with UV-C light within about 72 hours of harvesting,
curing the tobacco.

14. The method of claim 13, wherein the UV-C light has a peak intensity at about 254 nm.

15. The method of claim 13, wherein treating the freshly harvested tobacco with light UV-C light comprises exposing the freshly harvested tobacco to UV-C light having an intensity of between about 1 to 100 mW/cm² at the surface of the tobacco leaf for a period between about 1 minute to about 120 minutes.

16. The method of claim 13, wherein treating the freshly harvested tobacco with light UV-C light comprises exposing the freshly harvested tobacco to UV-C light having an intensity of between about 1 to 60 mW/cm² at the surface of the tobacco leaf for a period between about 10 minutes to about an hour.

17. The method of claim 13, further comprising permitting the freshly harvested tobacco to wilt for about a day prior to treatment with UV-C light.

18. The method of claim 13, wherein the curing step also comprises treating the tobacco with UV-C light one or more times more than about 72 hours after harvest.

19. A method of producing cured tobacco having reduced levels of TSNAs, the method comprising,
treating growing tobacco plants comprising leaves with a dose of UV-C light;
subsequently harvesting the tobacco; and,
curing the tobacco.

20. The method of claim 19, comprising treating the growing tobacco plants with UV-C light within about 1 week prior to harvesting.

21. The method of claim 19, comprising treating the growing tobacco plants with UV-C light within about 3 days prior to harvesting.

22. The method of claim 19, wherein the dose of UV-C light comprises exposure to about 1-60 mW/cm² for a period of about 15-60 minutes.

23. Cured tobacco produced according to the method of claim 1.

24. Cured tobacco produced according to the method of claim 13.

25. Cured tobacco produced according to the method of claim 19.

26. An apparatus comprising a chamber, one or more UV-C lights positioned therein, and a conveyor, wherein the conveyor is arranged to carry tobacco through the chamber and the lights are positioned so as to expose tobacco carried through the chamber on the conveyor to light produced by the UV-C lights.

27. The apparatus of claim 26, wherein the conveyor is constructed of a material that permits passage of a substantial amount of UV-C light therethrough and is arranged to move tobacco through the chamber and wherein two or more UV-C lights are positioned to simultaneously illuminate tobacco material from both sides of the conveyor.

28. An apparatus comprising a first chamber according to claim 26 and further comprising a second chamber comprising one or more UV-C lights and a conveyor, wherein the second chamber is arranged so that tobacco conveyed through the first chamber is subsequently conveyed through the second chamber.

29. An apparatus according to claim 28 wherein the conveyer of the first chamber and the conveyor of the second chamber are arranged so that tobacco is turned over between the first and second chambers.

30. An apparatus according to claim 26 wherein said conveyer is arranged to engage sticks on which tobacco plants have been hung and to convey the sticks into and out of the chamber.
